(19) United States
(12) Patent Application Publication
(10) Pub. No.: US 2014/0171664 A1
(43) Pub. Date: Jun. 19, 2014

(54) FURFURAL PRODUCTION FROM BIOMASS

(71) Applicants: Christopher Burket, Wilmington, DE (US); Keith W. Hutchenson, Lincoln University, PA (US)

(72) Inventors: Christopher Burket, Wilmington, DE (US); Keith W. Hutchenson, Lincoln University, PA (US)

(73) Assignee: E I DU PONT DE NEMOURS AND COMPANY, Wilmington, DE (US)

(21) Appl. No.: 14/238,202

(22) PCT Filed: Aug. 10, 2012
(86) PCT No.: PCT/US12/50473

§ 371 Date: Feb. 11, 2014

Related U.S. Application Data
(60) Provisional application No. 61/522,704, filed on Aug. 12, 2011.

(51) Int. Cl. C07D 307/50

(52) U.S. Cl. CPC C07D 307/50
USPC 549/489

(57) ABSTRACT

Furfural is produced from a xylan-containing lignocellulosic feedstock which is contacted with water in the presence of an acid catalyst. Specifically, the catalyst is sulfuric acid characterized by a room temperature pH in the range of about 0.2 to about 0.6. The use of sulfuric acid in place of phosphoric lowers costs and avoids the high viscosity of very low pH phosphoric acid.

### FIELD OF THE INVENTION

A method for the production of furfural from biomass is provided.

### BACKGROUND OF THE INVENTION

Furfural and related compounds are useful precursors and starting materials for industrial chemicals for use as pharmaceuticals, herbicides, stabilizers, and polymers. The current furfural manufacturing process utilizes biomass such as corn cob, switchgrass or wood waste as a raw material feed stock for obtaining xylose or hermicellulose. Furfural is derived from the hemicellulose fraction of lignocellulosic biomass as shown below:

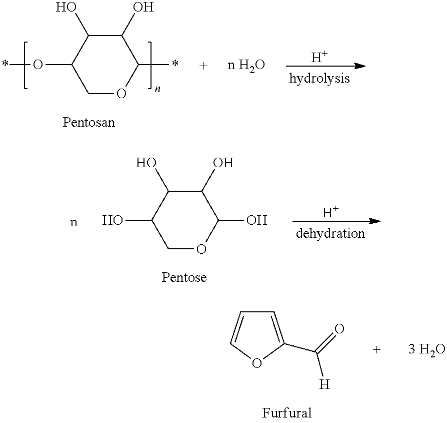

The hemicellulose, also referred to as xylan, pentosan, or C5, is hydrolyzed under acidic conditions to its monomeric form, which is referred to as xylose, pentose, or C5 sugar. In a similar environment, the sugar is subsequently dehydrated and cyclized to furfural. The rate of dehydration is an order of magnitude slower than hydrolysis.

A process for the manufacture of furfural, described in U.S. Pat. No. 6,743,928 (Zeitsch), includes the steps of charging a reactor with a pentosan (hemicellulose) containing material, heating the charge by introduction of pressurized steam to a first predetermined temperature, dosing the steam net valve of the reactor and opening a leak valve so as to produce a steady small flow of product vapor, thereby subjecting the charge to a gradual reduction of pressure until a second predetermined lower temperature is attained, the depressurization maintaining the liquid phase within the reactor in a constantly boiling state. Once the second temperature is reached, if no more furfural is obtained, the digestion is completed by opening another valve to discharge the residue. If, however, furfural is still being obtained, the reactor is reheated and submitted to another “gradual depressurization” period, (Abstract; col. 2, I. 32-50) Additional pressure/temperature cycles are carried out as deemed appropriate.

The pentosan-containing charge may or may not be acidified with an acid catalyst prior to heating. In the preferred form of the invention, phosphoric acid is the acid catalyst contacted with the raw material.” [col. 3, I. 7-8] Zeitsch explains this preference for phosphoric acid in K. J. Zeitsch, The Chemistry and Technology of Furfural and its Many By-Products; Elsevier: London, 2000, p. 61. “Depending on the primary temperature, the process can be run with or without a foreign acid. The higher the primary temperature, the smaller is the need for a foreign acid. If a foreign acid is used, it should not be sulfuric acid as the latter is known to cause some losses by sulfonation. On account of this effect, the “analytical furfural process”, having a yield of 100 percent with hydrochloric acid, does not give this theoretical yield when run with sulfuric acid. As in technical operations a use of hydrochloric acid would be inappropriate because of corrosion, and as nitric acid is out of the question because of nitration, the foreign acid of choice is orthophosphoric acid, since it does not cause any side reactions [40]. It is not a strong acid, but it is amply strong enough for the given purpose.” See also Arnold D. R., and Buzzard D. L. “A novel process for furfural production.” Proceedings of the South African Chemical Engineering Congress, 2003 3-5 Sep. 2003.

However, phosphoric acid presents cost, viscosity, and environmental issues. For example, it costs roughly an order of magnitude more than sulfuric acid. Also, highly acidic solutions, such as those having pH in the range of about 1 to 0, require a high enough wt % phosphoric acid that the resulting high viscosity poses additional processing problems. Therefore, a need remains for a more appropriate acid to catalyze this reaction that will work at least as well as phosphoric acid.

### BRIEF DESCRIPTION OF DRAWINGS

FIG. 1 is a schematic diagram of the apparatus used for Comparative Examples A and B and Examples 1-6.

### SUMMARY OF THE INVENTION

A process is provided for the production of furfural from biomass, comprising the steps of:
a) providing a lignocellulosic feedstock comprising xylan;
b) contacting the feedstock with aqueous sulfuric acid solution to form a reaction mixture in a reactor, wherein
   i) the room temperature pH of the aqueous sulfuric acid solution is in the range of about 0.2 to about 0.6, and
   ii) the liquid-to-solid ratio is in the range of about 0.1:1 to about 1:1 by weight;
c) heating the reaction mixture to a first predetermined temperature T₁ by introducing pressurized steam into the reactor; and
d) gradually reducing the pressure in the reactor until a second predetermined temperature T₂ is reached, wherein T₂ is lower than T₁, and wherein the rate of pressure reduction is sufficient to maintain liquid in the reactor in a constantly boiling state;
   whereby the xylan portion of the lignocellulosic feedstock is converted to furfural.

### DETAILED DESCRIPTION

### Definitions

The methods described herein are described with reference to the following terms.

As used herein, where the indefinite article “a” or “an” is used with respect to a statement or description of the presence of a step in a process of this invention, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

As used herein, the term “biomass” refers to any hemicellulosic or lignocellulosic material and includes materials comprising hemicellulose, and optionally further comprising cellulose, lignin, starch, oligosaccharides is and/or monosaccharides.

As used herein, the term “lignocellulosic” refers to a composition comprising both lignin and hemicellulose. Lignocellulosic material may also comprise cellulose.

All publications, patent applications, patents, and other references mentioned herein are incorporated by reference in their entirety. Unless otherwise defined, all technical and scientific terms used herein have the same meaning as commonly understood by one of ordinary skill in the art to which this invention belongs. In case of conflict, the present specification, including definitions, will control.

### Feedstock

In the processes described herein, a lignocellulosic feedstock comprising xylan is contacted with water in the presence of an acid catalyst, under suitable reaction conditions to form a mixture comprising furfural.

The source of the lignocellulosic feedstock is not determinative of the invention, and the biomass may be from any source. Biomass may be derived from a single source, or biomass can comprise a mixture derived from more than one source; for example, biomass could comprise a mixture of corn cobs and corn stover, or a mixture of grass and leaves. Biomass sources include, but are not limited to, bioenergy crops, agricultural residues, municipal solid waste, industrial solid waste, sludge from paper manufacture, yard waste, wood and forestry waste or a combination thereof. Examples of biomass include, but are not limited to, corn grain, corn cobs, crop residues such as corn husks, corn stover, grasses, wheat, wheat straw, barley, barley straw, hay, rice straw, cotton hulls, wild jujube shells, switchgrass, waste paper, sugar cane bagasse, sorghum, sweet sorghum stalk residue, palm oil empty fruit bunches, soy, components obtained from milling of grains, trees, branches, roots, leaves, wood chips, sawdust, shrubs and bushes, vegetables, fruits, flowers, and animal manure or a mixtures of at least two of these. Biomass that is useful for the invention may include biomass that has a relatively high carbohydrate value, is relatively dense, and/or is relatively easy to collect, transport, store and/or handle. In one embodiment of the invention, biomass that is useful includes corn cobs, wheat straw, sawdust, sorghum, sweet sorghum stalk residue, palm oil empty fruit bunches, cotton hulls, wild jujube shells, sugar cane bagasse, and mixtures of at least two of these.

The lignocellulosic feedstock may be used directly as obtained from the source, or energy may be applied to the biomass to reduce the size, increase the exposed surface area, and/or increase the availability of lignin, cellulose, hemicellulose, and/or oligosaccharides present in the biomass to the aqueous sulfuric acid solution. Energy means useful for reducing the size, increasing the exposed surface area, and/or increasing the availability of lignin, cellulose, hemicellulose, and/or oligosaccharides present in the lignocellulosic feedstock include, but are not limited to, milling, crushing, grinding, shredding, chopping, disc refining, ultrasound, and microwave. This application of energy may occur before and/or during contacting with the aqueous sulfuric acid solution. The lignocellulosic feedstock may be used directly as obtained from the source or may be dried to reduce the amount of moisture contained therein.

### Reaction Conditions

The lignocellulosic feedstock is contacted with aqueous sulfuric acid solution having a room temperature pH in the range of about 0.2 to about 0.6. The liquid-to-solid ratio is in the range of about 0.1:1 to about 1:1 by weight. In one embodiment, the liquid-to-solid ratio is in the range of about 0.4:1 to about 0.6:1. In one embodiment, an amount of solution is used which is at least equivalent to that of the lignocellulosic feedstock on a weight basis. Typically, the use of more water provides a more dilute solution of xylose (from hydrolysis of the xylan contained in the lignocellulosic biomass), which enables a higher overall yield of furfural to be realized. However, minimizing the amount of water used generally improves process economics by reducing process volumes. In practical terms, the amount of water used relative to the lignocellulosic feedstock will depend on the moisture content of the feedstock and on the desired yield of furfural, as well as the ability to provide sufficient mixing, or intimate contact, for the biomass hydrolysis and furfural production reactions to occur at a practical rate.

The first predetermined reaction temperature T₁ is in the range of about 220° C. to about 250° C. In one embodiment, T₁ is about 220° C. The second predetermined reaction temperature T₂ is in the range of about 170° C. to about 200° C. In one embodiment, T₂ is 170° C. or 200° C. In one embodiment, T₁ is 220° C. and T₂ is either 200° C. or 170° C. Larger differences between T₁ and T₂ can result in longer cycle time, which is defined as the time required to drop the temperature from T₁ to T₂ and then return to T₁. As the cycle time lengthens, a greater amount of time is spent purging at low temperatures and then reheating without purging furfural. Whenever the feedstock is at elevated temperature, furfural is generated and degraded; therefore, more frequent venting leads to higher yields. The heat-up time should also be minimized.

Suitable pressurization rates are between about 1 MPa/min and about 10 MPa/min. Suitable depressurization rates are between about 0.4 MPa/min and about 1 MPa/min. In one embodiment, the depressurization (pressure reduction) rate is about 0.4 to about 0.6 MPa/min. The rate of pressure reduction is sufficient to maintain liquid in the reactor in a constantly boiling state.

The number of cycles (T₁ to a temperature at about T₂ and then return to a temperature at about T₁) needed to obtain a high yield of furfural will depend upon the specific reaction conditions and is readily determined by one of ordinary skill in the art. In one embodiment, the number of cycles is 1, 2, 3, 4, 5, 6, 7, or 8.

Acid loadings, reaction temperatures, and cycle times will need to be optimized for each new feedstock introduced. For example, when corn stover and bagasse were tested as feedstocks at conditions where corn cob yielded ˜70% furfural, bagasse produced ˜63% and corn stover generated ˜43% furfural. The reaction conditions and biomass particle morphologies had not been not optimized for the two alternative feed stocks.

### EXAMPLES

The methods described herein are illustrated in the following examples. From the above discussion and these examples, one skilled in the art can ascertain the essential characteristics of this invention, and without departing from the spirit and scope thereof, can make various changes and modifications of the invention to adapt it to various uses and conditions.

### Materials

Corn cob was collected from one site of China Furfural Co., Ltd., Hebei Zhengtai Furfural plant. The corn cob was ground and sieved to take particles with size +12/−14 mesh. These particles were finally sealed in a plastic bag, and stored at room temperature until needed. The measured water content was 8.17 wt %, and the average composition, determined as described below, was (expressed as weight percent, dry basis): glucan, 19.03%; xylan, 27.28%; arabinan, 3.07%; acetyl groups, 2.23%.

Cotton hulls and wild jujube shells were also provided by China Furfural Co., Ltd., Hebei province. Cotton hull water content was 9.7 wt %. Cotton hull average composition (expressed as weight percent, dry basis) was: glucan, 19.7 wt %; xylan, 10.1 wt %; arabinan, 1.3 wt %. Wild jujube shell water content was 12.5 wt %. Wild jujube shell average composition (expressed as weight percent, dry basis) was: glucan, 19 wt %; xylan, 22.5% wt %; arabinan, 0.7 wt %.

Corn stover feed stock was provided by Nanjing Forest University. Water content was 8.17%, and average composition (expressed as weight percent, dry basis) was: glucan, 29%; xylan, 17.73%; arabinan, 3.09%.

Bagasse was courtesy of Jinan University. Water content was 8%, and average composition (expressed as weight percent, dry basis) was: glucan, 34.85%; xylan, 20.36%; arabinan, 1.79%.

Sweet sorghum stalk. residue was provided ZTE Energy Co., Ltd. (Beijing, China). The water content were 6.2 wt % and the average composition (expressed as weight percent, dry basis) was: glucan, 32.3 wt %; xylan, 20.3 wt %; arabinan, 1.6 wt %.

Sulfuric acid was made in Juzhou Juhua Reagent Co. Ltd, and purity was 95-98%. Phosphoric acid was produced from Guojia Jituan Chemical Reagent Co. Ltd, and its purity was not less than 85%.

[symbol]

### Methods

Apparatus

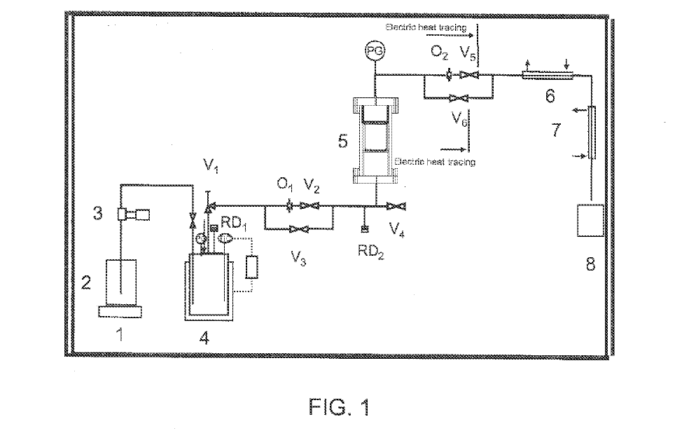

A schematic diagram of the apparatus is presented in FIG. 1. Its components included: a balance 1; a water glass bottle 2; a piston metering pump 3; a steam generator 4; a, reactor 5; coolers 6 and 7; a collector 8; 0.5 mm orifice plates O₁ and O₂; valves V₁-V₈; and rupture discs RD₁ and RD₂.

The apparatus basically consisted of four main parts: boner water feed system, steam generator, reactor, and coolers and cooling medium supply system. The steam generator 4 was an autoclave with a volume of 5 L and an outside electrical heater with a heating capacity of 3 KW. One temperature controller was fitted to control the liquid temperature by triggering the outside electrical heater. According to the total volume of the collected liquid in the collector 8, the pump 3 was started continuously or periodically to make up the same volume water into the steam generator 4 to maintain constant level in the reactor.

The reactor 5 was a fixed bed reactor which had double shells to avoid corn cob being singed. The corn cob particles were filled in the inner cylinder, which was about 106 mm high and whose ID was about 50 mm. There were two double-pipe coolers 6 and 7 in series . . . One was horizontal and another was vertical. Every cooler was about 400 mm long. The cooling media supply was the circulated 0° C. ethanol liquor, which was supplied by the refrigeration system.

Five thermocouples were respectively attached in the surface of the reactor inlet tube, bottom flange, reactor shell, upper flange and outlet pipe, and connected to the respective temperature controller to control the tracing temperature by triggering their respective outside electric belts. The connection tube was 6 mm ID 316L stainless steel. As for the reactor, the inlet tube, bottom flange, reactor outside, upper flange and outlet tube were all electrically traced and insulated.

Standard Operating Procedure

In general, feedstock particles (10 g or 16 g as indicated) were mixed with aqueous acid solution (liquid) at a liquid-to-solid ratio of 0.1:1, and then fed into the reactor. These temperature and pressure settings were used:
Steam generator liquid temperature: T₁+40° C. (but <270° C.)
Targeted trace temperature
Inlet tube: T₁+10° C.
Bottom flange: T₁+20° C.
Reactor shell: T₁+20° C.
Upper flange: T₁+20° C.
Outlet tube: T₁+10° C.

Reactor pressure p₀ during preheating: 2 berg (0.2 MPag) (p₂<6 berg), 6 berg (0.6 MPag) (p₂<6 berg).

For a cycling process, the reactor was heated to a temperature T₁ by introducing steam through an inlet valve, while the outlet valve was to closed. The inlet valve was closed and the outlet opened: vapor flashed from the reactor until a temperature T₂ was reached. The cycle was repeated by reheating the reactor to T₁. Vapor removed from the reactor was collected as condensate. Condensate from the reactor was collected and all reaction products analyzed.

Product Analysis

Reaction products were quantified via HPLC. The instrument was an HP 1100 Series with Agilent 1200 Series refractive index detector. The analytical method was adapted from an NREL procedure (NREUTP-510-42623). Both sugars and degradation products were measured on the same column, an Aminex® HPX-87H column from Bio-Rad Laboratories, Richmond, Calif. The mobile phase was 0.01 N H₂SO₄ flowing at 0.6 mL min⁻¹. The column temperature was 60° C. and the RI detector was set at 50° C. Samples were passed through a 0.2 μm filter before injection. The injection volume was 10 μL.

All yields are reported on a molar basis, where for the reaction xylan going to furfural, the yield is taken as the moles of furfural formed divided by the starting moles of xylan. Conversion is the moles of xylan reacted divided by the starting moles of xylan.

### Abbreviations

The meaning of abbreviations is as follows: “berg” means bar(s) gauge, “g” means gram(s), “HPLC” means high pressure liquid chromatography, “ID” means inner diameter, “KW” means kilowatt(s), “L” means liter(s), “min” means minute(s), “mL” means milliliter(s), means millimeter(s), “MPag” means megapascal(s) gauge, “N” means normal, “T” means temperature, “wt %” means weight percentage, “μL” means microliter(s), and “μm” means micrometer(s).

### Comparative Example A

### Cycling Process with pH 1 Phosphoric Acid

A mixture of corn cob (16 g) and pH 1 (6.7 wt % acid) aqueous phosphoric acid (6.4 g) was loaded into the reactor and subjected to a series of six temperature/pressure cycles. The liquid-to-solids ratio was 0.4:1. T₁ was 220° C. and T₂ was 170° C. The condensate was processed and analyzed as described above. The yield of furfural was 65%. Xylan conversion was essentially 100%.

### Comparative Example B

### Cycling Process with pH 1 Sulfuric Acid

A mixture of corn cob (16 g) and pH 1 (0.9 wt % acid) aqueous sulfuric acid (6.4 g) was loaded into the reactor and subjected to a series of six temperature/pressure cycles. The liquid-to-solids ratio was 0.4:1. T1 was 220° C. and T₂ was 170° C. The condensate was processed and analyzed as described above. The yield of furfural was 44%. Xylan conversion was essentially 100%.

### Example 1

### Cycling Process with pH 0 to 1 Sulfuric Acid

A mixture of corn cob (16 g) and aqueous sulfuric acid (6.4 g) at varying pH was loaded into the reactor and subjected to a series of six temperature/pressure cycles. T₁ was 220° C. and T₂ was 170° C. The liquid-to-solids ratio was 0.4:1. The condensate was processed and analyzed as described above. The furfural yields are reported in Table 1. Use of aqueous sulfuric acid in the range of pH 0.25-0.50 generated yields equivalent to pH 1 phosphoric acid in Camp. Ex. 1. In all runs, xylan conversion was essentially 100%,

TABLE 1

| Sulfuric Acid pH | Furfural Yield (%) |
|---|---|
| 0 | 48 |
| 0.13 | 54 |
| 0.25 | 67 |
| 0.37 | 67 |
| 0.50 | 68 |
| 0.62 | 56 |
| 0.75 | 52 |
| 1 | 41 |

### Example 2

### Cycling Process with Corn Stover Feedstock

A mixture of corn stover (10 g) and pH 0.37 aqueous sulfuric acid (4 g) was loaded into the reactor and subjected to a series of eight temperature/pressure cycles; reported yield was essentially achieved in six cycles. T₁ was 220° C. and T₂ was 170° C. The condensate was processed and analyzed as described above. The furfural yield was 44% and xylan conversion was 97%.

### Example 3

### Cycling Process with Bagasse Feedstock

A mixture of bagasse (10 g) and pH 0.37 aqueous sulfuric acid (4 g) was loaded into the reactor and subjected to a series of eight temperature/pressure cycles reported yield was essentially achieved in six cycles. T1 was 220° C. and T₂ was 170° C. The condensate was processed and analyzed as described above. The furfural yield was 64% and xylan conversion was 98%.

### Example 4

### Cycling Process with Cotton Hull Feedstock

A mixture of cotton hulls (10 g, having a dry basis analysis of 19.7 wt % glucan, 10.1 wt % xylan, and 1.3% arabinan) and pH 0.37 aqueous sulfuric acid was loaded into the reactor and subjected to a series of eight temperature/pressure cycles, reported yield was essentially achieved in six cycles. T₁ was 220° C. and T₂ was 170° C. The condensate was processed and analyzed as described above. The furfural yield was 15% and the xylan conversion was 95%.

### Example 5

### Cycling Process with Wild Jujube Skin Feedstock

A mixture of wild jujube skin feedstock (10 g, having a dry basis analysis of 19.0 wt % glucan, 22.5 wt % xylan and 0.7 wt % arabinan) and pH 0.37 aqueous sulfuric acid was loaded into the reactor and subjected to a series of eight temperature/pressure cycles, reported yield was essentially achieved in six cycles. T₁ was 220° C. and T2 was 170° C. The condensate was processed and analyzed as described above. The furfural yield was 62% and the xylan conversion was 99%.

### Example 6

### Cycling Process with Sweet Sorghum Stalk Residue Feedstock

A mixture of sweet sorghum stalk residue feedstock (10 g, having a dry basis analysis of 32.3 wt % glucan, 20.3 wt % xylan, and 1.6 wt % arabinan) and pH 0.37 aqueous sulfuric acid was loaded into the reactor and subjected to a series of eight temperature/pressure cycles, reported yield was essentially achieved in six cycles. T₁ was 220° C. and T₂ was 170° C. The condensate was processed and analyzed as described above. The furfural yield was 38% and the xylan conversion was 99%.

What is claimed is:

1. A process comprising the steps of:
a) providing a lignocellulosic feedstock comprising xylan;
b) contacting the feedstock with aqueous sulfuric acid to form a reaction mixture in a reactor, wherein
i) the room temperature pH of the aqueous sulfuric acid is in the range of about 0.2 to about 0.6, and
ii) the liquid-to-solid ratio is in the range of about 0.1:1 to about 1:1 by weight;
c) heating the reaction mixture to a first predetermined temperature T₁ by introducing pressurized steam into the reactor; and
d) gradually reducing the pressure in the reactor until a second predetermined temperature T₂ is reached, wherein T2 is lower than T₁, and wherein the rate of pressure reduction is sufficient to maintain liquid in the reactor in a constantly boiling state;
whereby the xylan portion of the lignocellulosic feedstock is converted to furfural.

2. The process according to claim 1, further comprising the sequential steps:
e) reheating the reaction mixture obtained in step d), to a temperature at about the first predetermined temperature T₁, then
f) gradually reducing the pressure in the reactor until a temperature at about the second predetermined temperature T₂ is reached, the rate of pressure reduction being sufficient to maintain liquid in the reactor in a constantly boiling state.

3. The process according to claim 2 wherein steps e) and f) are carried out sequentially 1 to 7 times.

4. The process according to claim 1 wherein the first predetermined temperature is in the range of about 220° C. to about 250° C.

5. The process according to claim 4 wherein the first predetermined temperature is about 220° C.

6. The process according to claim 1 wherein the second predetermined temperature is in the range of about 170° C. to about 200° C.

7. The process according to claim 6 wherein the second predetermined temperature is about 170° C. or about 200° C.

8. The process according to claim 7 wherein the first predetermined temperature is about 220° C.

9. The process according to claim 1 wherein the feedstock is corn grain, corn cobs, corn husks, corn stover, grasses, wheat, wheat straw, barley, barley straw, hay, rice straw, cotton hulls, wild jujube skin, switchgrass, waste paper, sugar cane bagasse, sorghum, sorghum stalk residue, palm oil empty fruit bunches, soy, components obtained from milling of grains, trees, branches, roots, leaves, wood chips, sawdust, shrubs, bushes, vegetables, fruits, flowers, or a mixture of at least two of these.

10. The process according to claim 8 wherein the feedstock is corn cobs, the first predetermined temperature is about 220° C. and the second predetermined temperature is about 170° C. or about 200° C.,
